(19) United States
(12) Patent Application Publication
(10) Pub. No.: US 2006/0239952 A1
(43) Pub. Date: Oct. 26, 2006

(54) Cleaning composition and method for preparing the same

(75) Inventor: Tatsuya Hattori, Kawasaki-shi (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: AJINOMOTO CO., INC., Tokyo (JP)

(21) Appl. No.: 11/393,679

(22) Filed: Mar. 31, 2006

Related U.S. Application Data
(63) Continuation of application No. PCT/JP04/14900, filed on Oct. 1, 2004.

(30) Foreign Application Priority Data
Oct. 3, 2003 (JP) 2003-345249

(51) Int. Cl. A61K 8/64; C07K 5/04

(52) U.S. Cl. 424/70.14; 530/331; 554/59

(57) ABSTRACT

Cleaning compositions comprising (A) N-long-chain acyl neutral amino acid or a salt thereof, (B) N-long-chain acyl neutral amino acid dipeptide or a salt thereof, and (C) N-long-chain acyl neutral amino acid tripeptide or a salt thereof provide abundant foam, are excellent in foam retaining properties, and furnish a cleaned and dried body or hair with both a smooth feeling and a moist feeling. Compositions which further comprise (D) a higher fatty acid having 8 to 22 carbon atoms or a salt thereof exhibit enhanced creamy properties in foam quality. N-long-chain acyl neutral amino acids or salts thereof may be conveniently prepared by mixing a neutral amino acid, a long-chain fatty acid, and at least one of an alkaline substance selected from sodium hydroxide and potassium hydroxide, and maintaining the mixture with heating, while removing the water produced during the reaction, with no catalyst being employed.

### CROSS REFERENCES TO RELATED APPLICATIONS

This application is a continuation of International Patent Application No. PCT/JP2004/014900, filed on Oct. 1, 2004, and claims priority to Japanese Patent Application No. 2003-345249, filed on Oct. 3, 2003, both of which are incorporated herein by reference in their entireties.

### BACKGROUND OF THE INVENTION

1. Field of the Invention

The present invention relates to cleaning compositions. The present invention also relates to methods for efficiently preparing such cleaning compositions and certain cleaning components contained in such cleaning compositions.

2. Discussion of the Background

An N-long-chain acyl neutral amino acid salt is a kind of N-long-chain acyl amino acid salt, and is used as a cleaning component for various cleaning compositions, since it has an excellent surface active effect and bacteriostatic effect, and has a low irritation to the skin.

However, when the N-long-chain acyl neutral amino acid salt is solely used as a cleaning agent, the resulting cleaning agent is likely to bring about such problems that it is insufficient in foamability such as lather amount and foam stabilizing property and is lacking in sensory feeling such as smooth feeling and moist feeling to be provided after cleaning and drying the body or hair. In order to improve those properties, various attempts have been made.

For example, with respect to an N-long-chain acylglycine, there has been disclosed a method wherein a small amount of an N-long-chain acylglycylglycine is added in order to improve its foam stabilizing property and sensory feeling (see, Japanese Patent No. 2,876,173). This method, however, has not yet achieved a satisfactory improvement to eliminate a tight feeling of the skin after cleaning, particularly after body cleaning, and is yet insufficient to give a sustainable moist feeling, though it has provided a little improvement.

Also, with respect to an N-long-chain acylalanine, similarly, it needs improving in foamability and smooth feeling for sensory feelings, though it provides a moist feeling, particularly after body cleaning. On the other hand, with respect to a surfactant such as an N-long-chain acylamino acid, and the like, there has been proposed a method wherein the surfactant is combined with a small amount of an N-long-chain acylalanylalanine salt, in order to provide a cleaning composition which is enhanced in foamability and foam stabilizing property, gives no creaky feeling upon cleaning, has an excellent resistance to hard water, and is excellent in washing-up feeling or wet feeling (see, Japanese Patent No. 3,296,062). The method, however, is insufficient in improvement of smooth feeling after body cleaning.

Therefore, there has been intensively desired a cleaning composition which comprises an N-long-chain acyl neutral amino acid or a salt thereof and will accomplish both foamability such as lather amount and foam stabilizing property and a smooth feeling and a moist feeling after drying.

On the other hand, in order to prepare an N-long-chain acyl neutral amino acid or the salt thereof, there has been known a method wherein an amino acid and a fatty acid halide are condensed or reacted with each other in an aqueous solution under an alkaline condition according to the Schotten-Bauman reaction, followed by taking up the reaction product as an acyl amino acid using an acid (see, for example, JP-A-05-70418 and JP-A-07-157795). This method, which is useful because the reaction is carried out under gentle conditions and proceeds for a relatively short period of time, has nevertheless problems that it requires an expensive fatty acid halide as the starting material and produces a large amount of inorganic salt as a by product which is not easy to remove by purification.

As an example of an attempt to avoid forming such salts as by-products, there may be mentioned U.S. Pat. No. 2,880,219, which discloses a method wherein a fatty acid is reacted with N-methyl taurine or taurine (aminosulfonic acids) to directly amidate. The reaction of the fatty acid with the aminosulfonic acid, however, requires the use of severe reaction condition of a high temperature of more than 200° C. for a long time.

U.S. Pat. No. 3,150,156 also discloses a method of preparing an acylmetyltaurine salt. In order to shorten the reaction time and to improve the reaction efficiency, various catalysts have been studied, and however, a lowered temperature of below 200° C. has been required to achieve a high yield.

On the other hand, JP-A-2002-234868 discloses a method of preparing an acyltaurine salt. In this method, a metal compound is used as a catalyst to react a fatty acid with a taurine salt, allowing the reaction time to be shortened, but the method can not avoid the use of a reaction temperature of beyond 190° C., and further, leaves a slight amount of the catalyst in the reaction product. Thus, the reaction mixture per se can not be used directly in a final product.

On the other hand, Published Japanese translation of WO96/39375 (Tokuhyo) No. 506743/'99 discloses, as an example of direct amidation of an amino acid with a fatty acid, a preparation method of the alkali metal salt of an N-acylamino acid, particularly sodium N-acylsarcosinate. This method is more specifically a method wherein the alkaline metal salt of an amino acid is directly reacted with a fatty acid at a high temperature while the water produced during the reaction is continuously removed. The method, however, must be carried out in a non-aqueous system from the outset of the reaction, and needs a complicated procedure such that the amino acid salt and free amino acid should be added individually. In addition, the method has a drawback that to increase the reaction rate and to achieve a high conversion rate require an excess of the free amino acid to be added in the reaction mixture of the alkali metal amino acid and the fatty acid, resulting in a surplus of the amino acid left after the reaction.

Under the above-described background art, there has been intensively desired a method for preparing an N-long-chain acyl neutral amino acid or a salt thereof, which method can proceed or be carried out under a relatively gentle reaction condition to provide a reaction mixture which can be used directly in a final product. There also remains a need for cleaning compositions which are free of the drawbacks described above.

### SUMMARY OF THE INVENTION

Accordingly, it is one object of the present invention to provide novel cleaning compositions.

It is another object of the present invention to provide novel cleaning compositions which give abundant foam.

It is another object of the present invention to provide novel cleaning compositions which provide a smooth feeling after cleaning and drying.

It is another object of the present invention to provide novel cleaning compositions which provide a moist feeling after cleaning and drying.

As used herein, the term “smooth feeling” means the smoothness felt when the hair or body is touched with the hand, and, as used herein, the term “moist feeling” means the state of the hair which is free from dryness or drying out, or state of the skin which feels fresh and soft.

It is another object of the present invention to provide novel methods for preparing an N-long-chain acyl neutral amino acid or a salt thereof.

It is another object of the present invention to provide novel, efficient, and simple methods for preparing an N-long-chain acyl neutral amino acid or a salt thereof.

These and other objects, which will become apparent during the following detailed description, have been achieved by the inventor's discovery that the following three components, that is, (A) an N-long-chain acyl neutral amino acid or a salt thereof, (B) an N-long-chain acyl neutral amino acid dipeptide or a salt thereof, and (C) an N-long-chain acyl neutral amino acid tripeptide or a salt thereof can be mixed at a specific weight ratio to provide a cleaning composition which brings about abundant foam, is excellent in foam stabilizing property, and furnishes a cleaned and dried body or hair with sensory feeling of both a smooth feeling and a moist feeling. Further, the inventor has found that (D) a higher fatty acid having 8 to 22 carbon atoms or a salt thereof may be added to those three components at a particular weight ratio to mix, thereby to provide a cleaning composition with an enhanced creamy property in foam quality.

Furthermore, the inventor has found that when a neutral amino acid, a long-chain fatty acid, and at least one alkaline substance selected from sodium hydroxide and potassium hydroxide are mixed, kept with heating, in an inert gas atmosphere if desired, and cleared continuously of the water produced during the reaction, a dehydration condensation reaction proceeds at a relatively low temperature in a relatively short time, resulting in the efficient production of an N-long-chain acyl neutral amino acid salt accompanied with no employed catalyst.

Thus, the present invention provides the following:

(1) A cleaning composition, comprising, as cleaning components:

(A) one or more N-long-chain acyl neutral amino acid or a salt thereof;

(B) one or more N-long-chain acyl neutral amino acid dipeptide or a salt thereof; and

(C) one or more N-long-chain acyl neutral amino acid tripeptide or a salt thereof, at a weight ratio of (A):(B):(C)=100:(8 to 50):(0.2 to 18).

(2) The cleaning composition of (1) above, wherein the weight ratio of the components is (A):(B):(C)=100:(9 to 45):(0.4 to 10).

(3) The cleaning composition of (1) or (2) above, which further comprises, as a cleaning component, (D) one or more higher fatty acid having 8 to 22 carbon atoms or a salt thereof.

(4) The cleaning composition of any one of (1) to (3) above, wherein the said cleaning composition is used for body cleaning.

(5) A method for preparing an N-long-chain acyl neutral amino acid salt, comprising:

(i) mixing three components of (a) a long-chain fatty acid, (b) a neutral amino acid, and (c) at least one alkaline substance selected from the group consisting of sodium hydroxide, potassium hydroxide, and mixtures thereof, to obtain a mixture; and

(ii) subjecting the mixture to heat-dehydration condensation reaction by heating at a maintained temperature of 150 to 190° C. while the water produced during the reaction is continuously removed.

(6) The method for preparing an N-long-chain acyl neutral amino acid salt of (5) above, wherein the said long-chain fatty acid is present in the mixture in an amount of 1.5 to 4 moles per one mole of the neutral amino acid.

(7) The method for preparing an N-long-chain acyl neutral amino acid salt of (5) or (6) above, wherein the heat-dehydration condensation reaction is carried out in an inert gas atmosphere.

(8) The method for preparing an N-long-chain acyl neutral amino acid salt of (5) to (7) above, wherein the alkaline substance is potassium hydroxide.

(9) A cleaning composition comprising, as the cleaning component, an N-long-chain acyl neutral amino acid salt prepared by the method of any one of (5) to (8) above.

(10) A cleaning composition comprising, as the cleaning component, the reaction mixture per se of an N-long-chain acyl neutral amino acid salt prepared by the method of any one of (5) to (8) above.

The present invention provides cleaning compositions which achieve an abundance of foam, exhibit excellent foam stabilizing properties, and furnish a cleaned and/or dried body or hair with sensory feelings of both a smooth feeling and a moist feeling. Further, the present invention provides cleaning compositions which exhibit an enhanced creamy property in foam quality. Furthermore, the present invention provides the preparation of N-long-chain acyl neutral amino acid salts efficiently with no employed catalyst through a dehydration condensation reaction which proceeds at a relatively low temperature over a short time. Therefore, it can provide a reaction mixture which can be used directly in a final product.

### DETAILED DESCRIPTION OF THE PREFERRED EMBODIMENTS

In the following, the present invention will be specifically explained.

Firstly, the cleaning composition of the present invention comprising, as cleaning components, three components (A), (B) and (C), or four components, (D) in addition to (A), (B) and (C) will be explained.

The acyl group of the components: (A) N-long-chain acyl neutral amino acid or a salt thereof, (B) N-long-chain acyl neutral amino acid dipeptide or a salt thereof, and (C) N-long-chain acyl neutral amino acid tripeptide or a salt thereof, which are used in the cleaning composition of the present invention, is a carbon chain which is derivable from a linear or branched alkyl or alkenyl fatty acid having 8 to 22 carbon atoms, and may be a single chain or mixed chains. Preferred examples are an octanoyl group, decanoyl group, lauroyl group, myristoyl group, palmitoyl group, stearoyl group, oleoyl group, coconut oil fatty acid acyl group, palm oil fatty acid acyl group, palm kernel oil fatty acid acyl group, and the like. Also, regarding an amino acid having an asymmetric carbon atom, it may be in the optically active form or the racemic form.

The neutral amino acid to be used as a starting material of the above-mentioned three components: (A) N-long-chain acyl neutral amino acid or a salt thereof, (B) N-long-chain acyl neutral amino acid dipeptide or a salt thereof, and (C) N-long-chain acyl neutral amino acid tripeptide or a salt thereof, of the cleaning composition of the present invention, may be one usually used and is not particularly limited. Usable examples are neutral amino acids having no optically active center (asymmetric carbon atom) such as glycine, β-alanine, and the like, neutral amino acids having an optically active center such as alanine, leucine, isoleucine, valine, threonine, proline, phenylalanine, and the like, and the like. These can be used alone or in the form of a mixture of two or more thereof. From the viewpoints of easy availability and processing and difficult decomposition, preferable are glycine, alanine, leucine, isoleucine, and valine, more preferable are glycine and alanine, and most preferable is glycine.

The cation of the salt-forming base component, which neutralizes the free carboxy group of the three components: (A) N-long-chain acyl neutral amino acid or a salt thereof, (B) N-long-chain acyl neutral amino acid dipeptide or a salt thereof, and (C) N-long-chain acyl neutral amino acid tripeptide or a salt thereof in the cleaning composition of the present invention to give their respective corresponding salts, may be one usually used and is not particularly limited. Usable examples are inorganic cations typically represented by the cations of alkaline metals such as sodium, potassium, and the like, and alkaline earth metals such as magnesium, calcium, and the like; and organic cations typically represented by cations of organic amines such as ammonium, alkanol aminium, and the like, protonated basic amino acids such as lysine, arginine, and the like. These can be used solely or in the form of a mixture of two or more thereof.

The method for preparing the components (A) N-long-chain acyl neutral amino acid or a salt thereof, (B) N-long-chain acyl neutral amino acid dipeptide or a salt thereof, and (C) N-long-chain acyl neutral amino acid tripeptide or a salt thereof in the cleaning composition of the present invention is not particularly limited. For example, the component (A) N-long-chain acyl neutral amino acid can be prepared according to a usual method such as the Schotten-Bauman reaction of an acyl neutral amino acid with a long-chain fatty acid chloride derivable from a long-chain fatty acid. The component (B) N-long-chain acyl neutral amino acid dipeptide or a salt thereof can be prepared according to a reaction such as the Schotten-Bauman reaction of an acid chloride derivable from (A) with a neutral amino acid. Similarly, the component (C) N-long-chain acyl neutral amino acid tripeptide or a salt thereof can also be prepared according to a reaction such as the Schotten-Bauman reaction of an acid chloride derivable from (B) with a neutral amino acid.

The total content of the three components (A) N-long-chain acyl neutral amino acid or a salt thereof, (B) N-long-chain acyl neutral amino acid dipeptide or a salt thereof, and (C) N-long-chain acyl neutral amino acid tripeptide or a salt thereof to be employed in the cleaning composition of the present invention is usually within a range of from 5 to 100% by weight, based on the total weight of the cleaning composition. From the viewpoint of achieving a sufficient effect, the total content of the three components is preferably from 7 to 95% by weight, and more preferably from 10 to 90% by weight. A total content of less than 5% by weight can not provide sufficient sensory feelings.

The weight ratio of the component (A):the component (B):the component (C) in the cleaning composition of the present invention is usually within a range of 100:(8 to 50):(0.2 to 18). In other words, component (B) is usually present in an amount of 8 to 50 parts by weight per 100 parts by weight of component (A), and component (C) is usually present in an amount of 0.2 to 18 parts by weight per 100 parts by weight of component (A). From the viewpoint of achieving a more distinct effect, the weight ratio of components (A):(B):(C) is preferably 100:(9 to 45):(0.4 to 10), more preferably 100:(10 to 40):(0.7 to 10), furthermore preferably 100:(12 to 35):(1 to 10), and particularly preferably 100:(14 to 25):(1.5 to 5). When component (B) and component (C) are present in an amount of less than 8 and less than 0.2 respectively, or in an amount of more than 50 and more than 18, the composition fails to provide sufficient effects in foamability upon cleaning the body or hair, and in smooth feeling and moist feeling after drying.

The acylamino acids (A), which are derivable from a neutral amino acid having no optical active center, inherently have the property of giving a smooth feeling or the like after drying, and can be added with the aforementioned given amount of (B), particularly the component (C) to bring about additionally a moist feeling after drying. As the particularly preferable neutral amino acid having no optical active center, glycine is mentioned from the viewpoint of easy availability and processing and high stability.

The acylamino acids (A), which are derivable from a neutral amino acid having an optical active center, inherently have the property of giving a moist feeling after drying, and can be added with the aforementioned given amount of (B), particularly the component (C) to bring about additionally a smooth feeling after drying. As the particularly preferable neutral amino acid having an optical active center, alanine is mentioned from the viewpoint of easy availability and processing and high stability.

Further, in addition thereto, (D) a higher fatty acid or a salt thereof, if desired, can be blended in the cleaning composition of the present invention to afford a creamy property.

The higher fatty acid to be blended in the cleaning composition of the present invention can be the same as the linear or branched alkyl or alkenyl fatty acid having 8 to 22 carbon atoms from which the aforementioned long-chain acyl group is derivable.

The component (D), a higher fatty acid or a salt thereof is usually blended to give a ratio relative to a total weight of the components (A), (B) and (C), of (A+B+C):(D)=100:(30 to 400), and preferably 100:(40 to 300) from the viewpoint of increasing creamy property in foam quality. In other words, component (D) may be added in an amount of 30 to 400 parts by weight per 100 parts by weight of the combined weight (A), (B), and (C) and preferably in an amount of 40 to 300 parts by weight per 100 parts by weight of the combined weigh of (A), (B), and (C). A ratio of less than 30 or more than 400 brings about insufficient creamy property in foam quality.

The cleaning composition thus prepared, of the present invention is usable for the hair and body, and, can be used, for example, as shampoos, rinsing shampoos, conditioning shampoos, facial cleansers, makeup removers, cleansing foams, cleansing powders, cleansing lotions, cleansing creams, hand soaps, bar soaps, mouth washes, shaving foams, body shampoos, and the like. Among them, it is preferably used for body.

Also, of course, there can be blended other component(s) usually used in cleaning compositions such as oil solutions, surfactants, gums, antiseptic agents, flagrance agents, UV absorbents, moisture holding agents, physiologically active components, antioxidants, anti-inflammatory agents, antibacterial agents, antiperspirants, chelating agents, neutralizing agents, pH regulating agents, and the like, in those cleaning composition, as far as they do not inhibit the effects of the present invention, according to exemplified uses and dosage forms.

The present cleaning compositions may be used to clean the hair or body (skin) by applying the cleaning composition to the hair or skin. The cleaning composition may be applied to dry hair and/or skin or wet hair and/or skin. The cleaning composition may be removed by rinsing with water and the hair and/or skin may then be dried.

Next, the method of preparation of the present invention and a cleaning composition using the reaction product obtained according to the preparation method are explained.

The preparation method of the present invention is a method wherein (a) a long-chain fatty acid, (b) a neutral amino acid, and (c) one or both of sodium hydroxide and potassium hydroxide are mixed and heated while the water produced during the reaction is removed continuously, thereby to obtain an N-long-chain acyl neutral amino acid salt, for example, an N-long-chain acylglycine salt. Herein, the continuous removal of the water produced means that both the water generated by neutralization with an alkaline substance and the water generated by condensation reaction shall be positively removed. Specifically, the removal can be achieved by removing together with an inert gas stream, or by removing under reduced pressure.

The long-chain fatty acid to be used as one of the starting materials in the preparing method of the present invention is a saturated or unsaturated linear or branched fatty acid having 8 to 22 carbon atoms. In this connection, this fatty acid can be a fatty acid of one kind, or a so-called mixed fatty acid of two or more kinds of fatty acids. Such a long-chain fatty acid can be the same as the above-mentioned linear or branched alkyl or alkenyl fatty acid having 8 to 22 carbon atoms which has already been explained in connection with the long-chain acyl to be derivable therefrom. Examples are, for instance, saturated linear fatty acids such as octanoic acid, decanoic acid, undecanoic acid, lauric acid, myristic acid, pentadecanoic acid, palmitic acid, stearic acid, behenic acid, and the like; unsaturated linear fatty acids such as oleic acid and linoleic acid; saturated branched fatty acids such as isostearic acid and the like; mixed fatty acids such as coconut oil fatty acid, palm oil fatty acid, and the like; and the like. These can be used alone or in the form of a mixture of two or more thereof. Among them, taking into consideration the stability to oxidation during the reaction, the saturated fatty acids are preferable. From the viewpoint of general purpose, particularly preferable are lauric acid, myristic acid, pentadecanoic acid, palmitic acid, and saturated mixed fatty acids containing these as the main component.

Another starting material used in the method of the present invention is a neutral amino acid, and there can be mentioned glycine, alanine, valine, leucine, isoleucine, phenylalanine, and the like. These can be used alone or in the form of a mixture of two or more thereof. The reason why the neutral amino acid is particularly employed among various amino acids is based on the thermal stability of the amino acid itself. Among them, from the viewpoint of excellent sensory of the reaction product, preferable are glycine and alanine, and particularly preferable is glycine.

The long-chain fatty acid and a neutral amino acid are used at a molar ratio of 1.5 to 4 moles of the long-chain fatty acid per one mole of the neutral amino acid, and can be used preferably at a molar ratio of 1.5 to 3 moles of the long-chain fatty acid per one mole of the neutral amino acid, from the viewpoint of cost and excellent sensory feelings of the reaction product. Further, from the viewpoint of improvement in reaction yield, the ratio is more preferably 2 to 3 moles of the long-chain fatty acid per one mole of the neutral amino acid. A surplus of fatty acid exists to liquefy the reaction mixture, but the reaction mixture, which has a small fatty acid content to lack in fluidity, if sufficiently blended before heating, allows the reaction to proceed in the solid state.

As the alkaline substance to be used in the preparation method of the present invention, any one or both of sodium hydroxide and potassium hydroxide can be used. Of the two alkaline substances, sodium hydroxide is preferable because of high reaction efficiency. The alkaline substance may be used as it is or in the form of an aqueous solution.

The alkaline substance is used in a molar ratio of equivalent or more to the neutral amino acid, from the viewpoint of reaction efficiency. No reaction takes place if no alkaline substance is used. On the other hand, using a surplus of the alkaline substance may cause uniform mixing to be difficult. Thus, a preferable molar ratio is 1 to 1.5 moles of the alkaline substance per 1 mole of the neutral amino acid. Moreover, taking into consideration the step for removing excess fatty acid directly from the reaction mixture by manipulations such as reduction of pressure and the like, the more preferable molar ratio is 1 to 1.3 moles of the alkaline substance per 1 mole of the neutral amino acid.

The reaction temperature for the dehydration condensation with heating in the preparation method of the present invention is 150 to 190° C., preferably 160 to 180° C. A temperature of less than 150° C. does not allow the reaction to proceed, or needs a lot of time to complete the reaction. A temperature of more than 190° C. is not necessary. The reaction is usually completed within 6 hours. A temperature of 160 to 180° C. takes about 2 hours to complete the reaction. The water produced during the reaction is removed continuously, as described above.

As the reaction condition for the preparation method of the present invention, the reaction mixture is preferably put in an atmosphere of an inert gas such as nitrogen, helium, or the like, in order to avoid coloration due to oxidation.

The N-long-chain acyl neutral amino acid salt prepared according to the preparation method of the present invention is in the form of the sodium salt and/or the potassium salt in the reaction mixture due to the used alkaline substance. The reaction mixture can be used as it is directly, or after being purified, as a cleaning component for various cleaning compositions. The purification method is not particularly limited, and a method conducted generally in this field can be utilized. For example, there is a method wherein the reaction is completed followed by removing excess fatty acid by distillation under a reduced pressure, or a method wherein the reaction mixture is neutralized with a mineral acid such as hydrochloric acid or sulfuric acid or the like to obtain the N-long-chain acyl neutral amino acid in the free form, and then an excess fatty acid and the like can be removed. In addition, the resulting free acid is neutralized with a proper base to prepare a desired N-long-chain acyl neutral amino acid salt. The reaction mixture is particularly preferably used directly as a cleaning component for various cleaning compositions, from the viewpoint that the reaction solution can be, as it is, a product accompanied with no waste solution to drain and can provide a cleaning composition which allows abundant foam upon cleaning and gives refreshing feeling and moist feeling after cleaning, particularly after body cleaning.

The cleaning composition, wherein such N-long-chain acyl neutral amino acid salt is used as a cleaning component, is usable for the hair and body in the same way as has been explained above, and can be used, for example, as shampoos, rinsing shampoos, conditioning shampoos, facial cleansers, makeup removers, cleansing foams, cleansing powders, cleansing lotions, cleansing creams, hand soaps, bar soaps, mouth washes, shaving foams, body shampoos, and the like. Among them, it is preferably used for the body.

Also, in the same way as has been explained above, various components usually used in cleaning compositions such as oil solutions, surfactants, gums, antiseptic agents, flagrance agents, UV absorbents, moisture holding agents, physiologically active components, antioxidants, anti-inflammatory agents, antibacterial agents, antiperspirants, chelating agents, neutralizing agents, pH regulating agents, and the like can be of course blended in those cleaning compositions, as far as they do not inhibit the effects of the present invention, according to exemplified uses and dosage forms.

Other features of the invention will become apparent in the course of the following descriptions of exemplary embodiments which are given for illustration of the invention and are not intended to be limiting thereof.

### EXAMPLES

### Examples 1 to 6 and Comparative Examples 1 to 3

Foaming and Sensory Evaluation of the Glycine Type.

Cleaning components were mixed at the proportions shown in Table 1 below, and then prepared into an aqueous solution containing a total content of 10% by weight of the components therein. Each of the aqueous solutions was diluted by 40 times (the cleaning components having a concentration of 0.25%), agitated with a mixer at a temperature of 30° C., and then measured for lather amounts at 1 minute and 5 minutes after the agitation. Foam retaining rate is indicated as the percentage of a lather amount at 5 minutes after the mixer agitation based on the lather amount at 1 minute after the mixer agitation. In Example 1, the reaction mixture obtained in Preparation Example 9 as mentioned hereinafter was cleared of the fatty acid to obtain a mixture of the acylamino acid and the acylpeptide. Both the acylamino acid and the acylpeptide were then converted to their respective sodium salts for use.

Lather amounts in Table 1 were evaluated on the basis of the lather amounts at 1 minute after the mixer agitation (1 minute lather amount), i.e., OO: 285 ml or more, O: 270 ml or more and less than 285 ml, Δ: 255 ml or more and less than 270 ml, and x: less than 255 ml. Foam stability was evaluated on the basis of foam retaining rate, i.e., OO: 80% or more, O: 75% or more and less than 80%, Δ: 65% or more and less than 75%, and x: less than 65%.

Foam quality and Feelings after dried in the same table were the results obtained by hand washing evaluation by five professional panelists. The evaluation was done by calculating an average value on the basis of the following standards (a) and (b), and an average of 4.5 or more was regarded as very good (OO), 3.5 to 4.4 was regarded as good (O), 2.5 to 3.4 was regarded as usual (Δ), 2.4 or less was regarded as bad (x).
(a) Foam quality (creamy)
   5: Very creamy
   4: Somewhat creamy
   3: Usual
   2: Somewhat rough
   1: Rough
(b) Smooth feeling after dried/Moist feeling after dried
   5: Very smooth/very moist
   4: Somewhat smooth/somewhat moist
   3: Usual
   2: Somewhat bad smooth feeling/somewhat bad moist feeling

1: No smooth feeling/no moist feeling

TABLE 1

| | | | | | | | Comp. | Comp. | Comp. |
|---|---|---|---|---|---|---|---|---|---|
| | Example 1 | Example 2 | Example 3 | Example 4 | Example 5 | Example 6 | Example 1 | Example 2 | Example 3 |
| Lauroyl | 100 | 100 | 100 | 100 | 100 | 100 | 100 | 100 | 100 |
| Gly Na | | | | | | | | | |
| Lauroyl | 15 | 15 | 34 | 15 | 20 | 25 | 0 | 6 | 55 |
| GlyGly Na | | | | | | | | | |
| Lauroyl | 2 | 10 | 1 | 2 | 5 | 7 | 0 | 0 | 20 |
| GlyGlyGly | | | | | | | | | |
| Na | | | | | | | | | |
| Lauric | 0 | 0 | 0 | 50 | 190 | 530 | 0 | 0 | 0 |
| acid Na | | | | | | | | | |
| 1 minute | 280 | 270 | 275 | 300 | 285 | 280 | 270 | 270 | 260 |
| lather | | | | | | | | | |
| amount | | | | | | | | | |
| (ml) | | | | | | | | | |
| 5 minute | 235 | 225 | 230 | 265 | 245 | 235 | 150 | 200 | 220 |
| lather | | | | | | | | | |
| amount | | | | | | | | | |
| (ml) | | | | | | | | | |
| Foam | 84 | 83 | 84 | 88 | 86 | 84 | 56 | 74 | 85 |
| retaining | | | | | | | | | |
| rate (%) | | | | | | | | | |
| Lather | ◯ | ◯ | ◯ | ◯◯ | ◯◯ | ◯ | ◯ | ◯ | Δ |
| amount | | | | | | | | | |
| evaluation | | | | | | | | | |
| Foam | ◯◯ | ◯◯ | ◯◯ | ◯◯ | ◯◯ | ◯◯ | X | Δ | ◯◯ |
| stability | | | | | | | | | |
| evaluation | | | | | | | | | |
| Foam | ◯ | ◯ | ◯ | ◯◯ | ◯◯ | ◯◯ | X | Δ | ◯ |
| quality | | | | | | | | | |
| (creamy) | | | | | | | | | |
| Smooth | ◯◯ | ◯◯ | ◯◯ | ◯◯ | ◯◯ | ◯◯ | ◯ | ◯◯ | ◯◯ |
| feeling | | | | | | | | | |
| after dried | | | | | | | | | |
| Moist | ◯◯ | ◯◯ | ◯◯ | ◯◯ | ◯◯ | ◯ | X | X | ◯◯ |
| feeling | | | | | | | | | |
| after dried | | | | | | | | | |

### Examples 7 to 12 and Comparative Examples 4 to 5

Foaming and Sensory Evaluation of the Alanine Type.

The alanine types were evaluated in the same manner. The results are shown in the following Table 2. In Table 2, the lather amount was evaluated on the basis of the lather amount of 1 minute after the mixer agitation, i.e., OO: 250 ml or more, O: 235 ml or more and less than 250 ml, Δ: 220 ml or more and less than 235 ml, and x: less than 220 ml. The foam stability was evaluated on the basis of the value of foam retaining rate, i.e., OO: 80% or more, O: 75% and less than 80%, Δ: 65% or more and less than 75%, and x: less than 65%. The sensory evaluation is the same as the evaluation method for the glycine types.

TABLE 2

| | | | | Example | Example | Example | Comp. | Comp. |
|---|---|---|---|---|---|---|---|---|
| | Example 7 | Example 8 | Example 9 | 10 | 11 | 12 | Example 4 | Example 5 |
| Lauroyl Ala K | 100 | 100 | 100 | 100 | 100 | 100 | 100 | 100 |
| Lauroyl | 8 | 20 | 35 | 8 | 20 | 20 | 0 | 4 |
| AlaAla K | | | | | | | | |
| Lauroyl | 0.3 | 0.5 | 2 | 0.3 | 0.5 | 0.5 | 0 | 0.1 |
| AlaAlaAla K | | | | | | | | |
| Lauric acid K | 0 | 0 | 0 | 90 | 160 | 325 | 0 | 0 |
| 1 minute | 240 | 255 | 245 | 245 | 255 | 250 | 220 | 230 |
| lather amount | | | | | | | | |
| (ml) | | | | | | | | |
| 5 minute | 200 | 210 | 205 | 200 | 205 | 200 | 140 | 180 |
| lather amount | | | | | | | | |
| (ml) | | | | | | | | |
| 10 minute | 60 | 60 | 60 | 180 | 190 | 170 | 40 | 40 |
| lather amount | | | | | | | | |
| (ml) | | | | | | | | |
| Form | 83 | 82 | 84 | 82 | 80 | 80 | 64 | 78 |
| retaining rate | | | | | | | | |
| (%) after 5 | | | | | | | | |
| minute | | | | | | | | |
| Form | 25 | 24 | 24 | 73 | 75 | 68 | 18 | 17 |
| retaining rate | | | | | | | | |
| (%) after 10 | | | | | | | | |
| minute | | | | | | | | |
| Lather | ◯ | ◯◯ | ◯ | ◯ | ◯◯ | ◯◯ | Δ | Δ |
| amount | | | | | | | | |
| evaluation | | | | | | | | |
| Foam stability | ◯◯ | ◯◯ | ◯◯ | ◯◯ | ◯◯ | ◯◯ | X | ◯ |
| evaluation | | | | | | | | |
| after 5 min. | | | | | | | | |
| Foam stability | X | X | X | Δ | ◯ | Δ | X | X |
| evaluation | | | | | | | | |
| after 10 min. | | | | | | | | |
| Foam quality | ◯ | ◯ | ◯ | ◯◯ | ◯◯ | ◯◯ | X | X |
| (creamy) | | | | | | | | |
| Smooth | ◯ | ◯◯ | ◯◯ | ◯ | ◯◯ | ◯◯ | Δ | Δ |
| feeling after | | | | | | | | |
| dried | | | | | | | | |
| Moist feeling | ◯ | ◯ | ◯◯ | ◯◯ | ◯◯ | ◯◯ | ◯ | ◯ |
| after dried | | | | | | | | |

### Preparation Example 1

5.0 g of glycine (66.7 mmole), 40.0 g of lauric acid (200 mmole), and 9.88 g of 27% sodium hydroxide solution (66.7 mmole) were mixed and then heated in a nitrogen gas stream at 180° C. for 1.5 hours. During the heating, the reaction mixture was in the state of liquid. The water produced during the reaction was continuously removed as water vapor together with the nitrogen gas stream. After cooling, a pale-yellowish solid was obtained. The reaction mixture was quantitatively determined by HPLC, regarding the acylated forms, i.e., the lauroylglycine and laurolyglycylglycine, the non-acylated forms, i.e., the glycylglycine and glycyldiketopiperazine, and the unreacted glycine. As a result, the reaction efficiency (yield of the acylated forms) was 91.8% by mole, and the overall recovering rate of the glycine present in the mixture (including the unreacted glycine) was 97.1%.

### Preparation Examples 2 to 11

Glycine or alanine, a fatty acid having a varying carbon chain length (different number of carbon atoms), and an alkaline substance shown in the following Table 3 were reacted in the same manner as in Preparation Example 1, except that the fatty acid and the alkaline substance were reacted in their various amounts shown (in molar ratio to the glycine or the alanine) and at the various reaction temperatures shown. The results are also shown in the following Table 3. It should be noted that the contents of the dipeptide and the tripeptide are shown as their relative weight values to the acylamino acid (the acylamino acid being assumed to be 100).

TABLE 3

| | | Preparation | Preparation | Preparation | Preparation | Preparation |
|---|---|---|---|---|---|---|
| | | Example 2 | Example 3 | Example 4 | Example 5 | Example 6 |
| Amino acid | | Glycine | Glycine | Glycine | Glycine | Glycine |
| Fatty acid | | | | | | |
| (Carbon atom number) | | C12 | C12 | C12 | C12 | C12 |
| (Equivalent) | | 3.0 | 3.0 | 3.0 | 3.0 | 2.5 |
| Alkali | | NaOH | NaOH | KOH | KOH | KOH |
| (Equivalent) | | 1.0 | 1.2 | 1.2 | 1.2 | 1.0 |
| Reaction temp. (° C.) | | 180 | 170 | 170 | 150 | 170 |
| Reaction time (hr) | | 1.5 | 1.5 | 1.5 | 6.0 | 1.5 |
| State of Reaction mixture | | liquid | liquid | liquid | liquid | liquid |
| Color of reaction product | | pale | pale | pale | pale | pale |
| | | yellowish | yellowish | yellowish | yellowish | yellowish |
| Reaction yield (%) | | 91.8 | 90.4 | 94.9 | 94.5 | 94.3 |
| Recovery yield(%) | | 97.1 | 97.0 | 98.2 | 98.0 | 96.4 |
| Content | Dipeptide | 24.0 | 18.0 | 18.0 | 18.0 | 22.7 |
| | Tripeptide | 3.5 | 2.2 | 2.3 | 1.7 | 3.2 |
| | | | | | Preparation | Preparation |
| | | Preparation | Preparation | Preparation | Example | Example |
| | | Example 7 | Example 8 | Example 9 | 10 | 11 |
| Amino acid | | Glycine | Glycine | Glycine | Alanine | Alanine |
| Fatty acid | | | | | | |
| (Carbon atom number) | | C14 | C12 | C12 | C12 | C12 |
| (Equivalent) | | 2.5 | 2.0 | 3.0 | 2.0 | 3.0 |
| Alkali | | KOH | KOH | KOH | KOH | KOH |
| (Equivalent) | | 1.2 | 1.2 | 1.4 | 1.4 | 1.4 |
| Reaction temp. (° C.) | | 170 | 180 | 170 | 185 | 180 |
| Reaction time (hr) | | 1.5 | 2.0 | 2.0 | 2.0 | 2.0 |
| State of Reaction mixture | | liquid | solid | liquid | liquid | liquid |
| Color of reaction product | | pale | pale | pale | pale | white |
| | | yellowish | yellowish | yellowish | yellowish | |
| Reaction yield (%) | | 94.0 | 90.8 | 95.0 | 92.0 | 94.5 |
| Recovery yield(%) | | 98.6 | 95.0 | 97.5 | 96.0 | 96.5 |
| Content | Dipeptide | 17.2 | 16.9 | 14.7 | 8.9 | 9.7 |
| | Tripeptide | 2.1 | 2.3 | 1.6 | 0.5 | 0.4 |

### Formulation Example 1

### Solid Cleaning Agent

A solid cleaning composition was prepared according to the formulation shown in the following Table 4. The solid cleaning agent thus obtained was excellent in foamability and evaluated by hand washing to provide performance to give both a moist feeling and a smooth feeling. The numerical values in the Table represent percents by weight.

TABLE 4

| | |
|---|---|
| Reaction product of Preparation | 35.0 |
| Example 6 | |
| Reaction product of Preparation | 25.0 |
| Example 7 | |
| Sodium N-stearoyl-L-glutamate | 15.0 |
| Sodium salt of coconut oil fatty acid | 5.0 |
| Cetanol | 5.0 |
| Glycerin | 5.0 |
| Sodium hydroxide | 3.5 |
| EDTA.2Na | 0.2 |
| Titanium oxide | 0.1 |
| Methylparaben | 0.1 |
| Water | remainder |
| Total | 100.0 |

### Formulation Example 2

### Cleansing Foam

An example of formulation of a cleansing foam is shown in the following Table 5. The numerical values in the Table represent percents by weight.

TABLE 5

| | |
|---|---|
| N-lauroylthreonine sodium salt | 10.0 |
| N-lauroylthreonylthreonine sodium salt | 2.0 |
| N-lauroylthreonylthreonylthreonine sodium salt | 0.3 |
| Sodium myristate | 8.0 |
| PEG2000 | 5.0 |
| Glycerin | 2.0 |
| Citric acid | 0.5 |
| Methylparaben | 0.2 |
| Water | remainder |
| Total | 100.0 |

### INDUSTRIAL APPLICABILITY

The present cleaning compositions may be used as shampoos, rinsing shampoos, conditioning shampoos, facial cleansers, makeup removers, cleansing foams, cleansing powders, cleansing lotions, cleansing creams, hand soaps, bar soaps, mouth washes, shaving foams, body shampoos, and the like, which can provide abundant foams, are excellent in foam stabilizing properties, and allow sensory feelings of both a smooth feeling and a moist feeling after cleaning and drying a body or hair.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A cleaning composition, comprising:
(A) one or more N-long-chain acyl neutral amino acid or a salt thereof;
(B) one or more N-long-chain acyl neutral amino acid dipeptide or a salt thereof; and
(C) one or more N-long-chain acyl neutral amino acid tripeptide or a salt thereof,
wherein said (A), (B), and (C) are present in a weight ratio of (A):(B):(C)=100: (8 to 50):(0.2 to 18).

2. The cleaning composition according to claim 1, which further comprises:
(D) one or more higher fatty acid having 8 to 22 carbon atoms or a salt thereof.

3. The cleaning composition according to claim 1, wherein said (A), (B), and (C) are present in a weight ratio of (A):(B):(C)=100:(9 to 45):(0.4 to 10).

4. The cleaning composition according to claim 3, which further comprises:
(D) one or more higher fatty acid having 8 to 22 carbon atoms or a salt thereof.

5. The cleaning composition according to claim 1, wherein:
said acyl in each of said (A), (B), and (C) is independently selected from the group consisting of a linear alkylcarboxyl group having 8 to 22 carbon atoms, a branched alkylcarboxyl group having 8 to 22 carbon atoms, a linear alkenylcarboxyl group having 8 to 22 carbon atoms, a branched alkenylcarboxyl group having 8 to 22 carbon atoms, and mixtures thereof; and
said neutral amino acid in each of said (A), (B), and (C) is independently selected from the group consisting of glycine, β-alanine, alanine, leucine, isoleucine, valine, threonine, proline, phenylalanine, and mixtures thereof.

6. The cleaning composition according to claim 2, wherein:
said acyl in each of said (A), (B), and (C) is independently selected from the group consisting of a linear alkylcarboxyl group having 8 to 22 carbon atoms, a branched alkylcarboxyl group having 8 to 22 carbon atoms, a linear alkenylcarboxyl group having 8 to 22 carbon atoms, a branched alkenylcarboxyl group having 8 to 22 carbon atoms, and mixtures thereof; and
said neutral amino acid in each of said (A), (B), and (C) is independently selected from the group consisting of glycine, β-alanine, alanine, leucine, isoleucine, valine, threonine, proline, phenylalanine, and mixtures thereof.

7. A method of cleaning skin or hair, comprising applying a cleaning composition according to claim 1 to skin or hair.

8. A method of cleaning skin or hair, comprising applying a cleaning composition according to claim 2 to skin or hair.

9. A method for preparing a cleaning composition according to claim 1, which comprises:
(i) mixing:
(a) a long-chain fatty acid;
(b) a neutral amino acid; and
(c) at least one alkaline substance selected from the group consisting of sodium hydroxide, potassium hydroxide, and mixtures thereof, to obtain a mixture; and
(ii) heating said mixture at a maintained temperature of 150 to 190° C. while continuously removing water produced to effect a heat-dehydration condensation reaction, to obtain a reaction mixture.

10. The method of claim 9, wherein said alkaline substance comprises potassium hydroxide.

11. The method of claim 9, wherein the said heating is carried out in an inert gas atmosphere.

12. The method of claim 11, wherein said alkaline substance comprises potassium hydroxide.

13. The method of claim 9, wherein said long-chain fatty acid is mixed with said neutral amino acid in an amount of 1.5 to 4 moles per one mole of said neutral amino acid.

14. The method of claim 13, wherein said alkaline substance comprises potassium hydroxide.

15. The method of claim 13, wherein the said heating is carried out in an inert gas atmosphere.

16. The method of claim 15, wherein said alkaline substance comprises potassium hydroxide.

17. A cleaning composition, comprising, said reaction mixture prepared according to claim 9.

18. A cleaning composition, comprising, said reaction mixture prepared according to claim 10.

19. A cleaning composition, comprising, said reaction mixture prepared according to claim 11.

20. A cleaning composition, comprising, said reaction mixture prepared according to claim 12.

21. A cleaning composition, comprising, said reaction mixture prepared according to claim 13.

22. A cleaning composition, comprising, said reaction mixture prepared according to claim 12.
